(19) United States
(12) Patent Application Publication
(10) Pub. No.: US 2025/0017517 A1
(43) Pub. Date: Jan. 16, 2025

(54) DETERMINING A TRIGGER LEVEL FOR A MONITORING ALGORITHM OF AN EPILEPTIC SEIZURE DETECTION APPARATUS

(71) Applicant: LIVASSURED B.V., Culemborg (NL)

(72) Inventors: Theo Ary Asmund TIELENS, Culemborg (NL); Michiel Robert Paul JANSEN, Culemborg (NL)

(21) Appl. No.: 18/713,234

(22) PCT Filed: Nov. 23, 2022
(86) PCT No.: PCT/EP2022/082910

§ 371 Date: May 24, 2024

(30) Foreign Application Priority Data
Nov. 26, 2021 (NL) 2029920

(51) Int. Cl. A61B 5/00

(52) U.S. Cl. CPC A61B 5/4094; A61B 5/7282

(57) ABSTRACT

A computer implemented method for determining a personalized trigger level based on which a monitoring algorithm of an epileptic seizure detection apparatus of a patient triggers an alarm. The monitoring algorithm evaluates a measurement signal for presence of a trigger level and triggers an alarm when the trigger level is detected in the measurement signal. The method uses a set of offline data of the patient, which data set includes marked true epileptic seizures of the patient. The method includes successively the steps a), b), c) and d). In step a) the set of offline data is input as the measurement signal into the monitoring algorithm, the offline data are processed with the monitoring algorithm, and, when the monitoring algorithm generates an alarm signal, a main-counter is increased by one, and a sub-counter is increased by one in case of a true alarm. In step b) an extent of false alarms is determined by comparing the sub-counter and main counter. Until the difference between the extent of false alarms and the predetermined value is within a predefined range, step c) successively and repeatedly: decreases the trigger level with a measure in case the extent of false alarms is below a predetermined value OR increases the trigger level with the measure in case the extent of false alarms is above the predetermined value; performs step a), and performs step b). In step d), the trigger level resulting from step c) is output as the personalized trigger level.

### FIELD OF THE INVENTION

The invention relates to the field of detection of epileptic seizures. More specifically, the invention is directed to determining the trigger level based on which a monitoring algorithm of an epileptic seizure apparatus triggers an alarm.

### BACKGROUND OF THE INVENTION

Sudden unexpected death of someone with epilepsy (SUDEP), may occur to a subject who was otherwise healthy. In SUDEP cases, no other cause of death is found when an autopsy is done. Each year, more than 1 out of 1,000 people with epilepsy die from SUDEP. If seizures are uncontrolled, the risk of SUDEP increases to more than 1 out of 150. These sudden deaths are rare in children but are known to be the leading cause of death in young adults with uncontrolled seizures.

The person with epilepsy is often found dead in bed, often in prone position. No one is sure about the cause of death in SUDEP. Some researchers consider the possibility that a seizure may cause an irregular heart rhythm. More recent studies have suggested that the person may suffocate from impaired breathing, fluid in the lungs, and being face down on the bedding (source Epilepsy Foundation USA website). The nine cases of SUDEP, that have been monitored accidentally, all happened within 1000 seconds after the end of the seizure.

The value of and/or changes in physiological parameters, such as heart rate, skin resistance, temperature, blood pressure, movement of limbs, etcetera may be an indication for a seizure. Wearable sensor devices for monitoring physiological parameters indicative for a seizure are known. Such devices can be worn on various body parts and limbs. Wearable sensor devices can for example be worn on the wrist. In other applications sensor devices may be worn on the upper arm.

Wearable sensor devices may have a physiological sensor such as a sensor measuring a heart activity, a blood pressure, a blood flow, a temperature, a skin resistance, movement, etcetera. An example of such a sensor is a photoplethysmographic sensor, which measures variations in light transmitted into a subject. Light from underlying tissue is reflected to a photosensitive sensor. The amount of reflected light depends on blood pressure. While the blood pressure varies in time with the subject's heart rate, the amount of reflected light thus allows a heart rate to be established accurately.

The established heart rate can be used in epileptic seizure detection. The heart rate often shows variations typical for an epileptic seizure. When an epileptic seizure is detected, especially during sleep, care takers such as medical staff, nurses but also partners of patients and/or parents may be notified using alarm systems communicatively coupled to detection devices. Thus, SUDEP may be prevented when such a care taker is duly notified allowing intervention when required especially in the 20 minutes after the actual seizure (source Brian J Dlouhy, Brian K Gehlbach and George B Richerson, 2015, Journal of Neurology, Neurosurgery & Psychiatry 2016 87:402-413).

Moreover, epileptic seizures may be detected using motion of the limbs.

Subjects suffering from epileptic seizures are actually most prone to SUDEP during the night lying in bed. Present epileptic seizure detecting devices suffer from false detections, related to postures deviating from lying down. In other circumstances however, detection may be required when the subject is incapacitated, and actually in an upright position not able to lie down.

Most of the severe epileptic seizures last between one and three minutes. That is the reason that most seizure detection and alarming devices strive to detect the seizure within 10-20 sec of the seizure onset. This short time frame in combination with the complexity of the condition and the behavior during epileptic seizures makes it up to date impossible to reach a sensitivity much better than 90% with the commonly accepted 50% false alarm rate.

The wish for a more reliable alarm is growing stronger especially since recent publications about the occurrence of Sudden Unexpected Death by Epilepsy indicate 3 SUDEPs every year out of 1000 people who suffer from epileptic seizures. Additionally, it becomes increasingly evident from publications that surveillance reduces the risk significantly, three times according to recent studies.

The invention aims at improving epileptic seizure detection apparatuses so that they have a better SUDEP warning than present epileptic seizure detection apparatuses. It is believed that SUDEP is especially caused or preceded by a severe seizure. Therefore, the invention further aims at improving epileptic seizure detection apparatuses so that they detect severe seizures more reliably in order to improve surveillance and thereby reduce SUDEP risk.

In general, the sensitivity of any detector may be improved by using more data, which translated to the field of epileptic seizure detecting apparatuses suggests a longer time interval of data collection. This has e.g. been proven by the offline data of the most extensive seizure detection trial worldwide, which was based on more than 30000 hours of nocturnal recordings of patients with epilepsy, see Johan Arends et al. Multimodal nocturnal seizure detection in a residential care setting: A long-term prospective trial, Neurology. 2018 Nov. 20; 91 (21): e2010-e2019. doi: 10.1212/WNL.0000000000006545. Epub 2018 Oct. 24. Neurology. 2018 Nov. 20; 91 (21): e2010-e2019. doi: 10.1212/WNL.0000000000006545. Epub 2018 Oct. 24. The detection algorithm performed better both in sensitivity and/or in positive predicting value, when the detection window was 40 seconds compared to the original algorithm with a 20 seconds window. An even longer detection window would further improve the sensitivity, since e.g. the heart rate accelerating effects of a seizure lasts much longer than of a normal nocturnal arousal, the latter of which is the most common cause of false alarms of an algorithm with a 20 seconds (or smaller) window. However, increasing the detection window result in less time being left for caretaker and doctors to react on an alarm and to act when necessary.

### SUMMARY OF THE INVENTION

It is an object of the invention to overcome one or more of the above problems. It is another object of the invention to improve the functioning of an epileptic seizure detection apparatus. A further object of the invention to improve the sensitivity of an epileptic seizure detection apparatus.

One or more of the above objects are according to a first aspect of the invention achieved by providing a computer implemented method for determining a personalized trigger level based on which a monitoring algorithm of an epileptic seizure detection apparatus of a patient—i.e. used or to be used by a patient-triggers an alarm, the monitoring algorithm being configured to evaluate a measurement signal for presence of a trigger level and to trigger an alarm signal when the trigger level is detected in the measurement signal;
wherein the method uses a set of offline data representative of the patient, which set of offline data:
   represents a measurement in time of one or more physiological parameters based on which the occurrence of an epileptic seizure can be determined, and
   comprises a multiple of marked true epileptic seizures;
wherein the method comprises successively the steps of:
a) inputting the set of offline data as the measurement signal into the monitoring algorithm, processing the offline data with the monitoring algorithm, and, when the monitoring algorithm triggers a said alarm signal:
   increasing a main-counter by 1, and
   increasing a sub-counter, which counts either true alarms or false alarms, by
      1 in case of a true alarm, respectively, a false alarm,
   wherein a said true alarm is an alarm associated to a said true epileptic seizure and a said false alarm is an alarm not associated to a said true epileptic seizure;
b) determining an extent of false alarms by comparing the sub-counter and main counter;
c) successively and repeatedly:
   decreasing the trigger level with a measure in case the extent of false alarms is below a predetermined value
   or
   increasing the trigger level with the measure in case the extent of false alarms is above the predetermined value,
   step a), and
   step b),
until the difference between the extent of false alarms and the predetermined value is within a predefined range having a lower limit and an upper limit; and
d) outputting the trigger level resulting from step c) as the personalized trigger level.

The offline data representative of the patient may be data of the patient him/herself, i.e. originating from the patient who will be (or is) the patient using the epileptic seizure apparatus having the monitoring algorithm with trigger level to be personalized. However, taking into account that it seems that specific patient conditions—like age and/or diabetes—may influence one or more of said physiological parameters based on which the occurrence of an epileptic seizure can be determined, it is also conceivable that ‘the offline data representative of the patient’ may be data of one or more patients having in common one or more said specific patient conditions—i.e. the specific patient conditions which may influence one or more of said physiological parameters based on which the occurrence of an epileptic seizure can be determined —. As the term ‘offline’ indicates these data have been collected and recorded earlier. The personalized trigger level is thus determined offline using offline data. These offline data may have been collected with the seizure detecting apparatus whose trigger level is to be personalized, but may also have been collected with another apparatus.

Epileptic seizure detecting apparatuses in general comprise:
a physiological sensor configured for measuring a physiological parameter of a patient and generating a measurement signal representative of the measured parameter, and
a processor configured to receive the measurement signal and provided with a monitoring algorithm configured to evaluate the measurement signal for presence of a trigger level and to trigger an alarm signal when the trigger level is present in the measurement signal.

The detecting apparatus and its monitoring algorithm are carefully checked and calibrated by the manufacturer of the seizure detecting apparatus. This in order to ensure a correct functioning of the seizure detecting apparatus. Incorrect functioning or malfunctioning may result in dead of a user of the seizure detecting apparatus. Similar missing a seizure—i.e. not detecting a seizure-results in not triggering an alarm signal, which may result in dead of a user as well. For this reason, the manufacturers provide the seizure detecting apparatus in general with a default trigger level for the monitoring algorithm which has a fixed setting, i.e. the default trigger level cannot be changed.

According to the invention, the trigger level is personalized using a set of offline data representative of the patient. This personalizing may start from a trigger level having basically any value when starting the method according to the invention. With a new seizure detection apparatus the trigger level may, at the start of the method according to the invention, have a default value as set by the manufacturer. With a used seizure detection apparatus, the trigger level may, at the start of the method according to the invention, have a personalized value of a previous patient or of the same patient. In the latter case, the trigger level at the start of the method may be, for example, an earlier personalized one which is to be (further) finetuned, a personalized value from another apparatus, or an estimated one.

The offline data comprise a multiple of marked ‘true epileptic seizures’ of the said patient. ‘True epileptic seizure’ does not necessary mean that the patient actually had that seizure, but it means that this part of data is assumed to represent a (true) epileptic seizure. The marking of these ‘true epileptic seizures’ may have been done manually for example by a data annalist. Alternatively or additionally, this marking may have been done in automated manner by using one or more (different) algorithms through which the offline data are processed or have been processed.

The markings of the true epileptic seizures in the off line data may have an accuracy or reliability which is superior or highly superior to the accuracy or reliability of the monitoring algorithm to be improved/personalized. Using offline data with such superior markings of true epileptic seizures, results in so to say training the monitoring algorithm to become more accurate or reliable in triggering its alarms, i.e. less false alarms. The personalizing of the trigger level is done by feeding the algorithm of the trigger level to be personalized with the offline data instead of with real time data as obtained during normal monitoring use of the epileptic seizure detection apparatus. Each time the algorithm triggers an alarm in response to the offline data: i) a main-counter is increased with one, and ii) a sub-counter is increased with one only in case the respective alarm in response to the offline data is caused by a true epileptic seizure in the offline data (or alternatively is increased with one only in case the respective alarm in response to the offline data is not caused by a true epileptic seizure in the offline data). Subsequently, an extent of false alarms is determined by comparing the sub-counter and main-counter (or alternatively an extent of true alarms may be determined by comparing the main-counter and sub-counter). Subsequently, the trigger level is, depending on the extent of true/false alarms in relation to the total of alarms, increased or decreased with a measure, followed by:
resetting the main-counter and sub-counter,
again feeding the offline data through the algorithm with accordingly adjusted trigger level,
again increasing the main-counter in case of an alarm and increasing the sub-counter in case of a true alarm or false alarm;
again determining an extent of false alarms (or true alarms) by comparing the main-counter and sub-counter, and
and repeating these steps until the difference between the extent of false alarms and the predetermined value is within a predefined range.

In other words this ‘personalizing’ is an iterative optimization of the trigger level, which iterative process is stopped once a criterion (i.e. the difference between the extent of false alarms and the predetermined value being with in a predetermined range) is met. This iterative process may basically be any iterative process suitable for optimizing the trigger level. The measure of increase or decrease may for example be a constant/fixed value or a varying value. In case of a varying value, the measure may for example decrease with each iterative step or it may be a function of the difference between the extent of false (or true) alarms and the predetermined value. The larger the difference the larger the measure may be.

Once the extent of false (or true) alarms is within the predetermined range, the resulting trigger level is outputted as the personalized trigger level. This outputted personalized trigger then may be entered or stored as trigger level to be used when real-time monitoring a patient.

The physiological parameter based on which the occurrence of an epileptic seizure can be determined, may according to the invention comprise one or more of: heart rate of the patient, electrical heart activity of the patient, blood pressure variation of the patent, blood flow, skin resistance of the patient, temperature of the patient, movement of the patient, movement of a body part of the patient, etc.

In a monitoring algorithm of an epileptic seizure detection apparatus, the sensitivity of the epileptic seizure detection apparatus will increase when the trigger level is decreased, and the sensitivity of the epileptic seizure detection apparatus will decrease when the trigger level is increased.

According to a further embodiment of the first aspect of the invention, the lower limit may be about or equal to 0.95 times the predetermined value, such as about or equal to 0.97 times the predetermined value or about or equal to 0.989 times the predetermined value. In other words, the predefined range may be [X-5%, Y], such as [X-3%, Y] or [X-1.1%, Y], with X being the predetermined value and Y being the upper limit.

According to another further embodiment of the first aspect of the invention, the upper limit may be about or equal to 1.05 times the predetermined value, such as about or equal to 1.03 times the predetermined value OR about or equal to 1.011 times the predetermined value. In other words, the predefined range may be [Q, R+5%], such as [Q, R+3%] or [Q, R+1, 1%], with R being the predetermined value and Q being the lower limit. Alternatively, the upper limit may according to an alternative further embodiment be about or equal to the predetermined value.

For example, the predefined range may be [T−5%, T+5%] or [T−3%, T+3%] or [T−1.1%, T+1.1%], with T being the predetermined value, or according to another example the predefined range may be [T−5%, T] or [T−3%, T] or [T−1.1%, T] with (again) T being the predetermined value.

According to another further embodiment of the first aspect of the invention, step c) consists of either a step c1) in case the extent of false alarms is below the predetermined value, or a step c2) in case the extent of false alarms is equal to or above the predetermined value; wherein step c1) comprises successively and repeatedly:
decreasing the trigger level with the measure,
step a), and
step b),
   until the extent of false alarms exceeds above the predetermined value; and
   wherein step c2) comprises successively and repeatedly:
increasing the trigger level with the measure,
step a), and
step b),
   until the extent of false alarms drops below the predetermined value.

With this iterative process in which step c) consists of either step c1) or step c2), the rate of false alarms is:
either
larger than the predetermined vale and approaches the predetermined value from above in which case the entire iterative process is according to step c2,
or
smaller than the predetermined value and approaches the predetermined value from below, in which case the entire iterative process is according to step c1.
In case the entire iterative process is according to step c1 and in case the predefined range has as its upper limit the predetermined value, the last part of step c1)—i.e. after the extent of false alarms has exceeded the predetermined value—may be lowering the trigger level such that the extent of false alarms falls within the predefined range.

With this iterative process in which step c) consists of either step c1) or step c2), the measure may especially be a small percentage of the trigger level. The measure may for example be in the range of 0.5% to 5% of the trigger level, such as in the range of about 0.5 to 2.5% of the trigger level. The measure may for example be about 1% or about 0.5% of the trigger level.

According to another further embodiment of the first aspect of the invention, the extent of false alarms is the percentage of alarms which is a said false alarm. The predetermined value then may be smaller than or equal to 50% or the predetermined value may be in the range of 30% to 50%.

According to another further embodiment of the first aspect of the invention, the extent of false alarms may the number of false alarms within a period of time. For example the limit for acceptable false alarms could be one false alarm per four nights.

In case of raw offline data, i.e. offline data having not yet marked true epileptic seizures, the method according to the first aspect of the invention, may in a further embodiment comprise an additional step x) of processing the offline data and marking epileptic seizures found in this offline data as true epileptic seizures, the additional step x) taking place before step a).

In case of raw offline data, step x) may use a computer implemented, first seizure detecting algorithm to find epileptic seizures in the offline data and to mark the epileptic seizures detected by the first seizure detecting algorithm as said true epileptic seizures, and wherein the first seizure detecting algorithm is different from the monitoring algorithm. As the raw offline data may comprise thousands of hours of recorded data, a computer implemented seizure detecting algorithm allows a large reduction of costs compared to marking the true epileptic seizures manually by humans. Not only because a computer implemented algorithm can process the data faster—for example by accelerated playback of the recording—but also because computer time in general is much cheaper than human time. The algorithm used for marking the raw data—in this embodiment called the first seizure detecting algorithm—may in a further embodiment be an algorithm which is typically superior—such as in accuracy—to/over the monitoring algorithm which trigger level is to be improved/personalized. Due to being superior this algorithm used for marking the raw data (the first seizure detecting algorithm) is inherently slower than the monitoring algorithm which trigger level is to be improved/personalized.

In case the offline data already have been provided with marked ‘true epileptic seizures’, the true epileptic seizures may, according to a further embodiment of the first aspect of the invention, be seizures detected as an epileptic seizure by a first seizure detecting algorithm which has processed the offline data and has marked the epileptic seizures detected by the first seizure detecting algorithm as said true epileptic seizures, and wherein the first seizure detecting algorithm is different from the monitoring algorithm. As said before, the algorithm used for marking the raw data—in this embodiment called the first seizure detecting algorithm—may in a further embodiment be an algorithm which is typically superior—such as in accuracy—to/over the monitoring algorithm which trigger level is to be improved/personalized. Due to being superior this algorithm used for marking the raw data (the first seizure detecting algorithm) is inherently slower than the monitoring algorithm which trigger level is to be improved/personalized.

In case of raw offline data the step x) may, according to another embodiment of the invention, comprise the sub-steps of:
processing the offline data with a computer implemented, first seizure detecting algorithm to find seizures in the offline data,
processing the offline data with a computer implemented, second seizure detecting algorithm to find seizures in the offline data, wherein the second seizure detecting algorithm is different from the first seizure detecting algorithm,
comparing the seizures detected by the first seizure detecting algorithm with the seizures detected by the second seizure detecting algorithm and marking an event present in the offline data as a true epileptic seizure when the first seizure detecting algorithm as well as the second seizure detecting algorithm have detected this event as an epileptic seizure.

Doing so, two different seizure detecting algorithms are used for accurately determining and marking the so called ‘true epileptic seizures’.

In case the offline data already have been provided with marked ‘true epileptic seizures’, the true epileptic seizures may, according to a further embodiment of the first aspect of the invention, be seizures:
detected as an epileptic seizure by a first seizure detecting algorithm which has processed the offline data and a second seizure detecting algorithm which has processed the offline data, and
marked as a said true epileptic seizure when detected by both the first seizure detecting algorithm and the second seizure detecting algorithm; and
   wherein the first seizure detecting algorithm being different from the second seizure detecting algorithm. As two different seizure detecting algorithms have been used for determining, the marking of the so called ‘true epileptic seizures’ will be more accurately.

In case the ‘true epileptic seizures’ are obtained by using a first seizure detecting algorithm and a second seizure detecting algorithm, the first seizure detecting algorithm may, according to a further embodiment of the first aspect of the invention, be a slow algorithm detecting a seizure on the basis of a change in the physiological parameter during a first period of time, whilst the second seizure detecting algorithm is a fast algorithm detecting a seizure on the basis of a change in the physiological parameter during a second period of time, the second period of time being shorter than the first period of time. Doing so the accuracy in the marking of ‘true epileptic seizures’ in the offline data may be increased by combining the benefits of a fast and slow seizure detecting algorithm. In a further embodiment, the first period of time may be at least two times, such as at least 5 or 10 times, as long as the second period of time. With respect to a ‘slow algorithm’ and ‘fast algorithm’ it is noted that slow and fast may be in relation to the length of the time window taken into account by the algorithm to determine whether the physiological parameter(s) observed show in that window sign of a seizure. An algorithm using a small window of time will be faster than an algorithm using a larger window of time. However, slow and fast more in general the terms slow and fast relate to the time the algorithm requires to do its detection task. A complex algorithm using a time window of 10 seconds may for example take 1 minute to do its detection-so called detection time-, whilst a simple algorithm using a window of 10 seconds may take about 10 seconds. According to a further embodiment of the invention the slow seizure detection algorithm may have a detection time of 40 seconds or more, such as 2 minutes or more. The fast seizure detection algorithm may have a detection time of at most 20 seconds, such as in the range of 10-20 seconds or less than 10 seconds.

According to another further embodiment of the first aspect of the invention, in which embodiment a first seizure detecting algorithm and a second seizure detecting algorithm are used or have been used for marking the ‘true epileptic seizures’ in the offline date, the second seizure detecting algorithm may be the same as the monitoring algorithm. Doing so, the accuracy of a monitoring algorithm, in general a relatively inaccurate fast algorithm with a small detection window, can be improved by means of another slower algorithm in the form of the first seizure detecting algorithm with high accuracy.

According to another further embodiment of the first aspect of the invention, the method may comprise a step e) of setting the personalized trigger level as the trigger level of the monitoring algorithm for online (or real time) use on a patient.

According to another further embodiment of the first aspect of the invention, the computer may comprise a data output port configured for outputting the personalized trigger level and/or a data input port for receiving the offline data.

One or more of the above objects are according to a second aspect of the invention achieved by providing a trigger level determining apparatus for determining a personalized trigger level based on which a monitoring algorithm of an epileptic seizure detection apparatus of a patient triggers an alarm, in which the trigger level determining apparatus being configured for carrying out the method according to the first aspect of the invention. Alternatively worded, the trigger level determining apparatus thus may comprise a processor configured to perform the method according to the first aspect of the invention. This trigger level determining apparatus may according to the invention be an apparatus separate from the epileptic seizure apparatus using the monitoring algorithm with personalized trigger level real time on a patient.

The trigger level determining apparatus may according to a third aspect of the invention also be incorporated in an epileptic seizure monitoring apparatus. Accordingly, one or more of the above objects are according to the third aspect of the invention achieved by providing an epileptic seizure detection apparatus, wherein the apparatus comprises:
a physiological sensor configured for measuring a physiological parameter of a patient and generating a measurement signal representative of the measured parameter,
a processor configured to receive the measurement signal and provided with a monitoring algorithm configured to evaluate the measurement signal for presence of a trigger level and to trigger an alarm signal when the trigger level is present in the measurement signal, and
a trigger level entry configured for receiving, as the trigger level to be used in the monitoring algorithm, a personalized trigger level obtained by the method according to the first aspect of the invention.

According to another further embodiment of the third aspect of the invention, the apparatus may further comprises a storage configured to receive and store the measurement signal as offline patient data, and the processor may further configured to perform the method according to the first aspect of the invention using the offline data stored in the storage. With this embodiment, the epileptic seizure monitoring apparatus may be used with an initial trigger level—for example a default trigger level set by the manufacturer of the epileptic seizure monitoring apparatus—until the offline patient data stored in the storage contain a sufficient number of marked ‘true epileptic seizures’—for example at least 50, at least 100 or more ‘true epileptic seizures’—, and then determine a personalized trigger level according to the method of the first aspect of the invent, and subsequently offline adjusting the monitoring algorithm of the epileptic seizure monitoring apparatus by entering the thus obtained personalized trigger level in the trigger level entry.

One or more of the above objects are according to a fourth aspect of the invention achieved by providing:
a computer program comprising instructions which, when the program is executed by a computer, cause the computer to carry out the steps of the method according to the first aspect of the invention;
or
a computer program product comprising instructions which, when the program is executed by a computer, cause the computer to carry out the steps of the method according to the first aspect of the invention.

One or more of the above objects are according to a fifth aspect of the invention achieved by providing a computer readable data carrier having stored thereon a computer program or computer program product according to the fourth aspect of the invention.

One or more of the above objects are according to a sixth aspect of the invention achieved by providing a data stream which is representative of a computer program or a computer program product according to the fourth aspect of the invention.

In relation to the term ‘true seizure’ detected and marked by a seizure detecting algorithm, as used in this application, it is to be noted that the detection algorithm detects pieces of data in the offline data that may be an indication of a real seizure, but this is not sure. No diagnoses has been made. The piece of data detected in the offline data may actually not represent a real seizure. In case the seizure detection algorithm marks a seizure (piece of data) as a true seizure, it is thus an assumption, a working hypothesis, that it is a real seizure. For this reason it is called a ‘true seizure’.

### BRIEF DESCRIPTION OF THE DRAWING

The invention will be explained further with reference to the drawings. In these drawings:

FIG. 1 shows, schematically and in perspective, an epileptic seizure detecting apparatus according to the invention;

FIG. 2 shows a patient carrying the epileptic seizure detecting apparatus according to the invention;

FIG. 3 shows in a schematic diagram an first embodiment of the method according to the invention; and

FIG. 4 shows in a schematic diagram a second embodiment of the method according to the invention.

### DETAILED DESCRIPTION OF THE EMBODIMENTS

Embodiments of the invention will be elucidated in the description below with reference to the drawings.

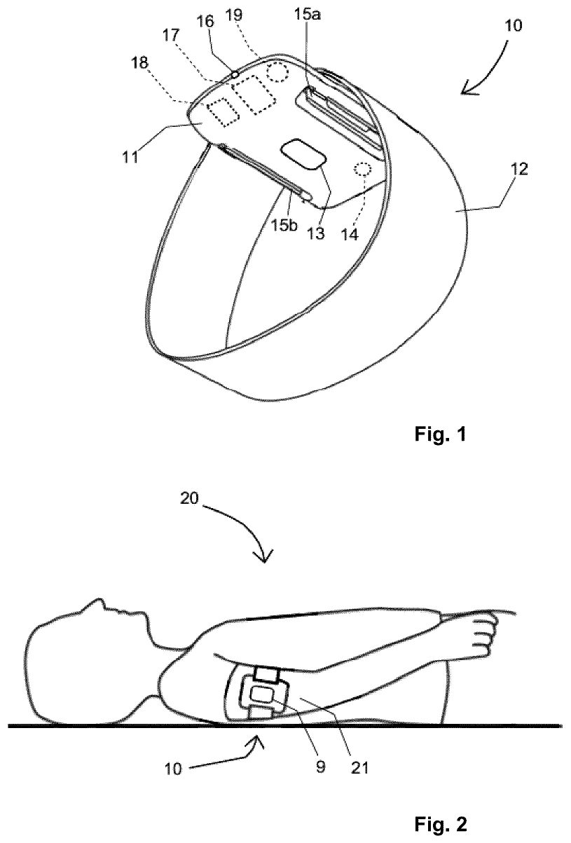

FIG. 1 shows an epileptic seizure detection apparatus 10 according to the invention. The apparatus 10 comprises a housing 11, and a band 12 attached to the housing 11. The band 12 attached to the housing 11 allows the epileptic seizure detection apparatus 10 to be worn by a patient. The apparatus according to the invention may for example be worn around an arm, for example an upper arm, around a leg, for example an upper leg, or around the chest. The band 12 may be of rigid material, of elastic material, or of a rigid material with an elastic portion. The housing 11 may be made of a plastic. The housing 11 is provided with fasteners 15a, 15b for attaching the band 12 to the housing 11.

The epileptic seizure detection apparatus 10 is equipped with at least one physiological sensor, such as a first physiological sensor 13 and/or a second physiological sensor 14.

The physiological sensor 13 may be a heart activity sensor, however other physiological sensor types such as EEG electrodes, ECG electrode blood pressure, motion detectors-like for example the physiological sensor 14 to be discussed further below-, temperature, respiration, etc. may be used to establish respective physiological parameters which may be used to detect epileptic seizure.

The physiological sensor 13 may comprise a photoplethysmographic sensor which operates by sending light into the skin of the subject wearing the epileptic seizure detection apparatus 10. The photoplethysmographic sensor is, in this example, located in a center region of the baseplate 16 of the housing 11. Any light reflected from within the skin of the subject, is detected by a photo detector comprised in the photoplethysmographic sensor 13. Variations in the detected reflected light provide a measurement signal representative of the heart activity, which measurement signal allows a heartrate of the patient wearing the device to be established. The measurement signal may also be provided by means of an sensor formed by an electrode pair for measuring heart activity signal electrically, i.e. electrocardiography, which measurement signal also allows the heart rate of the patient to be established.

As known in the field of epileptic seizure detection, an epileptic seizure can be seen in the heart activity of the patient, for example in the heart rate of the patient. The heart activity of the patient already shows characteristics associated to epileptic seizure some time before the epileptic seizure becomes or can become a severe one. In case the heart activity is monitored by the monitoring algorithm, the trigger level determines when a monitored characteristic in the measurement signal representing the heart activity is of a level that an alarm signal is to be triggered. The lower the trigger level, the higher the sensitivity is and thus the greater the chance of a false alarm. The higher the trigger level, the lower the sensitivity is and thus the greater the chance that a severe epileptic seizure is missed.

The epileptic seizure detection apparatus 100 of this example, may further be provided with second physiological sensor 14 accommodated within the housing 11 and depicted as a dotted circle.

Such a second physiological sensor may for example be movement sensor, like an acceleration sensor 14, providing a measurement signal representing the movement, like acceleration, of the body part of the patient to which it is mounted.

As known in the field of epileptic seizure detection—see for example WO-2020/043270, an epileptic seizure can be seen in movements of body part of a patient, for example in movement of a limb, like an arm, of a patient. These movements already show characteristics associated to epileptic seizure some time before the epileptic seizure becomes or can become a severe one. In case the movement of a body part is monitored by the monitoring algorithm, the trigger level determines when a monitored characteristic in the measurement signal representing the movement of a body part of a patient is of a level that an alarm signal is to be triggered. The lower the trigger level, the higher the sensitivity is and thus the greater the chance of a false alarm. The higher the trigger level, the lower the sensitivity is and thus the greater the chance that a severe epileptic seizure is missed.

Referring to FIG. 1, the epileptic seizure detecting apparatus according to the invention may further comprises a computer 17, for example a processor 17, and a battery 19 for feeding the computer, the first physiological sensor 13, and second physiological sensor 14 with electricity. The battery may be a rechargeable battery, which may be charged wireless or by wired connection.

Referring to FIG. 1, the epileptic seizure detecting apparatus according to the invention may optionally comprise a port 16 configured for uploading and/or downloading data obtained by or to be used by, respectively, the epileptic seizure detecting apparatus 10. This port 16 may be either a wired port or a non-wired port, like a Bluetooth port.

In case the personalized trigger level is determined on a separate device external from the epileptic seizure detecting apparatus 10, this port 16 may optionally be used as trigger level entry for entering the personalized trigger level into the monitoring algorithm of the epileptic seizure detecting apparatus 10. But, as will become clear further below, the trigger level entry is not required to be a port for external communication between the epileptic seizure detecting apparatus 10 and the surrounding. When the method for determining a personalized trigger level is integrated in the computer/processor 17, then the trigger level entry may be any internal communication inside the epileptic seizure detecting apparatus 10 feeding the personalized trigger level obtained with the method according to the invention as trigger level to be used into the monitoring algorithm.

This port 16 may optionally also be configured for feeding the epileptic seizure detecting apparatus with electricity from external source, in which case the battery may be dispensed with. This electricity may also be used for charging the battery in case not dispensed with.

Referring to FIG. 1, the epileptic seizure detecting apparatus 10 according to the invention may optionally further comprise a storage 18 for storing data. The epileptic seizure detecting apparatus 10, may for example be used for obtaining the set of offline data used by storing the measurement signal. The epileptic seizure detecting apparatus may for example be taken in use with a default trigger level set by the manufacturing, and then, after having collected and stored sufficient offline data representative of the patient, the personalized trigger level may be determined offline—i.e. whilst the epileptic seizure detecting apparatus is not in use for live (online) detecting epileptic seizure of the patient, in said differently while the epileptic seizure detecting apparatus is not in use on the patient—. After having offline determined the personalized trigger level, this personalized trigger level will be used as the trigger level in future/next live/online runs of the monitoring algorithm on the patient. The method according to the invention may also use as a starting point for the trigger level an earlier determined personalized trigger level. This may for example be useful to further fine tune the personalized trigger level with a larger or new set of offline data representative of the patient.

As will be clear to the skilled man, the epileptic seizure detection apparatus 10 may also be without a band 12. The epileptic seizure detection apparatus 10 may be only a housing of whatever shape, which housing is provided with one or more of the above-described components. It is for example conceivable that the housing has no fasteners 15a, 15b for a band and that a band separate from the housing or tape is used to mount the housing on the patient. Further it is also conceivable that the epileptic seizure detecting apparatus functions from a distance without contacting the patient, i.e. without any band or adhesive being necessary.

FIG. 2 schematically shows a patient 20 lying in a bed and wearing an epileptic seizure detection apparatus 10 according to the invention on his upper arm 21. As indicated with 9, the epileptic seizure detection apparatus 10 may optionally be provided with a screen 9 allowing a visual read out of—for example—the measurement signal, the status of the patient, or the cause of an alarm.

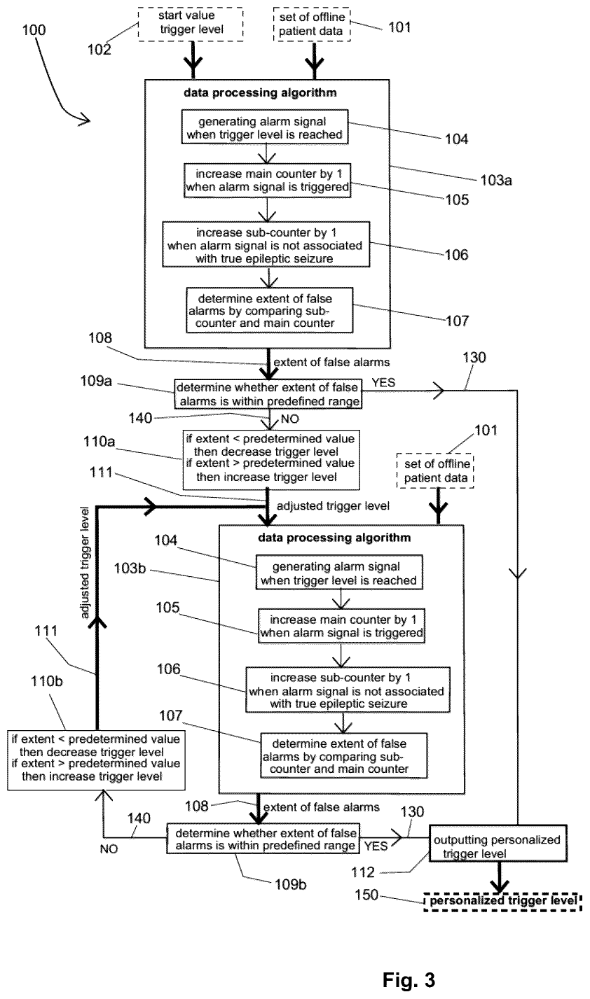

FIG. 3 schematically shows the method according to the invention.

In FIG. 3, the blocks with solid lines represent steps in the method, the blocks with dotted lines represent input used by the method and the end output resulting from the method. Further, thin arrowed lines represent a direction to continue from one step to the other step. Of these thin arrowed lines, the ones to be followed in case the preceding step results in a YES carry the reference number 130, the ones to be followed in case the preceding step results in a NO carry the reference number 140, and the remaining ones have no reference number.

Further in FIG. 3, the bold arrowed lines represent ‘input to’ and ‘output from’ a step in the method.

As shown in FIG. 3, the computer implemented method 100 for determining a personalized trigger level has two inputs: a ‘set of offline patient data’ 101 and ‘a start value for the trigger level’ 102. This trigger level, also abbreviated as TL, may be a default trigger level as set by the manufacturer, an earlier obtained personalized trigger level, or any other value usable as trigger level.

These two inputs are fed into the ‘data processing algorithm’ 103a which basically represents step a) and step b) of the method.

In step a), the data processing algorithm 103a feeds the offline data through the monitoring algorithm of the epileptic seizure apparatus in which the personalized trigger level obtained with the method according to the invention is intended to be used. When encountering an epileptic seizure in the offline data (i.e. the trigger level is reached), the monitoring algorithm will generate an alarm signal, see the block 104. Each time an alarm is triggered, a main counter mc is increased by one (i.e. mcₙ=mcₙ₋₁+1), see the block 105. As this alarm may be a true alarm or a false alarm, the data processing algorithm looks whether the alarm signal was triggered by an event which has been marked as a true epileptic seizure in the offline data. In case the alarm signal has been triggered by a true epileptic seizure, a sub counter sc is increased by one (i.e. scₙ=scₙ₋₁+1), see block 106. Alternatively, it is also conceivable to increase the sub-counter for each false alarm (instead of for each true alarm).

After the entire set of offline data has been fed through the monitoring algorithm step b) of the method follows. The data processing algorithm 103a determines in block 107—step b)—the extent of false alarms by comparing the sub-counter and main counter.

The extent of false alarms may for example be defined as a percentage of the total of alarms, i.e. sc/mc expressed in %. It is however also possible to define the extent of false alarms differently, for example by defining a false alarm rate per unit of time. An example of this may be one or two false alarms per four nights being acceptable and more than one or two, respectively, being not acceptable.

This extent of false alarms is fed as input 108 to next step 109a. In this next step it is determined whether the extent of false alarms is within a predefined range relative to the predetermined value, which is the same as determining whether the difference between the extent of false alarms and the predetermined value—i.e. extent of false alarms minus the predetermined value (or the other way around)—is within a predefined range. If so the start value for the trigger level is maintained as personalized trigger level and outputted in step 112 as personalized trigger level 150. As will be clear to the skilled man, the step 109 is, as at this stage of the method according to the invention, optional and has therefore been indicated as 109a in order to differentiate from a basically same step 109—indicated as 109b—further down in the method according to the invention.

In case the extent of false alarms is not within the predefined range relative to the predetermined value, the extent of false alarms is passed to the next step 110a. In step 110a the trigger level is adjusted. In step 110a, the trigger level will be decreased in case the extent of false alarms is smaller than the predetermined value, or the trigger level will be increased in case the extent of false alarms is larger than the predetermined value. In case, step 109a might be absent one of these two if criteria may in addition contain condition ‘equal to’ as well, resulting in ‘the trigger level will be increased in case the extent of false alarms is larger than or equal to the predetermined value’ or, alternatively, ‘the trigger level will be decreased in case the extent of false alarms is smaller than or equal to the predetermined value’.

Subsequently, the trigger level adjusted in step 110a and the set of offline patient data is used are used as inputs for a next the data processing algorithm 103b, which is basically identical to the earlier described data processing algorithm 103a. Therefore, for further explanation of the data processing algorithm 103b, reference is made to the above elucidation of date processing algorithm 103a.

Like with the data processing algorithm 103a, also the output of data processing algorithm 103b is an extent of false alarms 108. This extent of false alarms is passed to a next step 109b, which is basically identical to the step 109a described above. The difference being that step 109b is not optional, whilst step 109a may be optional. In case, in step 109b the extent of false alarms is within the predefined range, the trigger level used as input for the last run of the data processing algorithm 103b is passed to the step 112 for outputting it as personalized trigger level. In case, in step 109b the extent of false alarms is not within the predefined range, step 110b follows in which the trigger level is adjusted in the same manner as in step 110a-described above. Subsequently, the trigger level adjusted in step 103b and the set of offline patient data 101 are used in a next run of the data processing algorithm 103b, etcetera.

With respect to steps 109a and 109b it is noted that the predefined ranges of both steps may be the same or different, and that the upper and lower limit of these predefined ranges may be fixed values or may be variable values.

It is further noted that the increase or decrease of the trigger level in steps 110a and 110b will be with a measure, which measure may vary—for example depending on how far the extent of false alarms is away from the predefined range—or may be a fixed measure.

The main-counter and sub-counter may be reset to zero before each run of the data processing algorithm 103a and 103b. However, it is also conceivable that no reset takes place, for example in case the starting values of the main-counter and sub-counter at the start of each run of data processing algorithm 103a and 103b are taken into account as well.

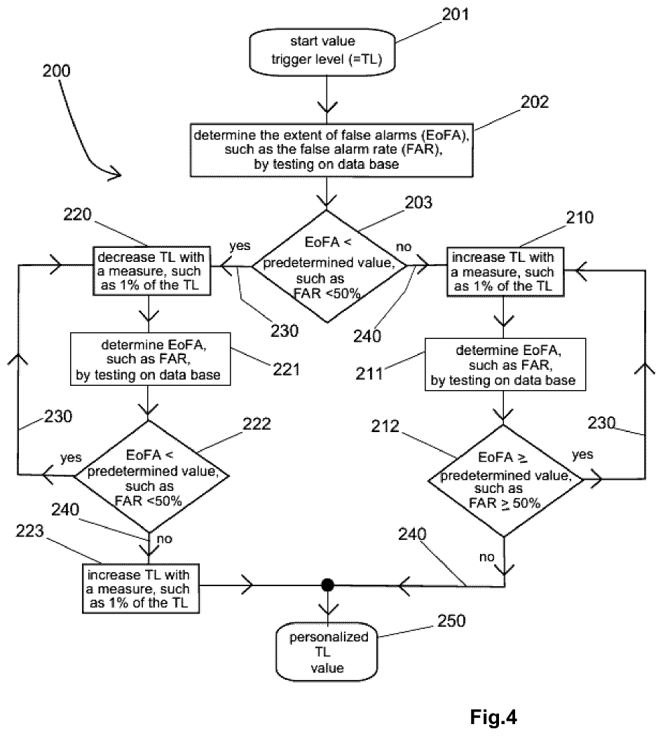

Now turning to FIG. 4, an example of a further embodiment is schematically shown.

In FIG. 4, only the last step 107 of the data processing algorithm 103a has been shown as step 202 for reasons of simplicity. The steps 104, 105, and 106 of the data processing algorithm 103a may be present as well in the method of FIG. 4 as preceding step 202 of FIG. 4. In FIG. 4, the step 109a of FIG. 3 is absent and the step 203 is the equivalent of step 110a of FIG. 3.

In the FIG. 4 embodiment, the trigger level is decreased with a measure when the extent of false alarm rates is smaller than a predetermined value or increased with a measure in case the extent of false alarm rates is larger than or equal to the predetermined value.

The embodiment of FIG. 3 may result in the embodiment of FIG. 4 when the measure used to increase or decrease the trigger level in steps 110a and 110b of FIG. 3 is relatively small compared to the momentaneous trigger level which is to be increased or decreased in step 110a or 110b. What is relatively small with respect to the momentaneous trigger level, may depend from circumstances like the type of physiological parameter measured, the allowable extent of false alarms, the predetermined value, the limits of the predefined range, and other circumstances. In practise, a measure of 0% to 5% of the momentaneous trigger level, such as in the range of 0.5 to 5% or in the range 0.5 to 2.5%, like about 0.5 or about 1%, turns out to be relatively small compared to the momentaneous trigger level.

With the embodiment of FIG. 4, the extent of false alarms will iteratively approach the predetermined value from basically either below (i.e. the extent of false alarms is lower than the predetermined value and stepwise approaches the predetermined value with each iteration) or above (i.e. the extent of false alarms is higher than the predetermined value and stepwise approaches the predetermined value with each iteration).

In FIG. 4, the steps 221 and 211 are mutually basically about the same, and are in turn the same as step 107 in processing algorithm 103b of FIG. 3. Also here it is to be noted that in FIG. 4, only the last step 107 of the data processing algorithm 103b has been shown as step 211 and 221 for reasons of simplicity. The steps 104, 105, and 106 of the data processing algorithm 103b may be present as well in the method of FIG. 4 as preceding steps 211 and 221, respectively.

As in epileptic seizure detection, it may be desirable to ensure that the extent of false alarms is on the end not below the predetermined value, step 223 may optionally be added to adjust the trigger level in this respect.

Embodiments and further embodiments of the present invention may be expressed in words as set out in the following clauses:

Clause 1: A computer implemented method for determining a personalized trigger level based on which a monitoring algorithm of an epileptic seizure detection apparatus of a patient triggers an alarm, the monitoring algorithm being configured to evaluate a measurement signal for presence of a trigger level and to trigger an alarm signal when the trigger level is detected in the measurement signal;
wherein the method uses a set of offline data representative of the patient, which set of offline data:
   represents a measurement in time of at least one physiological parameter based on which the occurrence of an epileptic seizure can be determined, and
   comprises a multiple of marked true epileptic seizures;
wherein the method comprises successively the steps of:
   a) inputting the set of offline data as the measurement signal into the monitoring algorithm, processing the offline data with the monitoring algorithm, and, when the monitoring algorithm triggers a said alarm signal:
      increasing a main-counter by 1, and
      increasing a sub-counter, which counts either true alarms or false alarms, by 1 in case of a true alarm, respectively, a false alarm,
wherein a said true alarm is an alarm associated to a said true epileptic seizure and a said false alarm is an alarm not associated to a said true epileptic seizure;
   b) determining an extent of false alarms by comparing the sub-counter and main counter;
   c) successively and repeatedly:
      decreasing the trigger level with a measure in case the extent of false alarms is below a predetermined value
   or
   increasing the trigger level with the measure in case the extent of false alarms is above the predetermined value,
      step a), and
      step b),
until the difference between the extent of false alarms and the predetermined value is within a predefined range having a lower limit and an upper limit; and
   d) outputting the trigger level resulting from step c) as the personalized trigger level.

Clause 2: Method according to clause 1, wherein the lower limit is about or equal to 0.95 times the predetermined value, such as about or equal to 0.97 times the predetermined value or about or equal to 0.989 times the predetermined value.

Clause 3: Method according to one of the preceding clauses, wherein the upper limit is about or equal to 1.05 times the predetermined value, such as about or equal to 1.03 times the predetermined value or about or equal to 1.011 times the predetermined value.

Clause 4: Method according to one of the clauses 1-2, wherein the upper limit is about or equal to the predetermined value.

Clause 5: Method according to one of the preceding clauses, wherein step c) consists of either a step c1) in case the extent of false alarms is below the predetermined value, or a step c2) in case the extent of false alarms is equal to or above the predetermined value;
wherein step c1) comprises successively and repeatedly:
   decreasing the trigger level with the measure,
   step a), and
   step b),
until the extent of false alarms exceeds above the predetermined value; and
wherein step c2) comprises successively and repeatedly:
   increasing the trigger level with the measure,
   step a), and
   step b),
until the extent of false alarms drops below the predetermined value.

Clause 6: Method according to clause 5 in combination with clause 4, wherein in step c1), after the extent of false alarms has exceeded the predetermined value, the trigger level is lowered such that the extent of false alarms falls within the predefined range.

Clause 7: Method according to one of the preceding clauses, wherein the measure is a percentage of the trigger level.

Clause 8: Method according to clause 7, wherein the measure is in the range of 0.5% to 5%, such as about 1%, of the trigger level.

Clause 9: Method according to one of the preceding clauses, wherein the extent of false alarms is the percentage of alarms which is a said false alarm.

Clause 10: Method according to clause 9, wherein the predetermined value is smaller than or equal to 50%.

Clause 11: Method according to clause 10, wherein the predetermined value is in the range of 30% to 50%.

Clause 12: Method according to one of the clauses 1-8, wherein the extent of false alarms is the number of false alarms within a period of time.

Clause 13: Method according to one of the clauses 1-12, wherein the method comprises an additional step x) of processing the offline data and marking epileptic seizures found in this offline data as true epileptic seizures, and wherein the additional step x) takes place before step a).

Clause 14: Method according to clause 13, wherein the step x) uses a computer implemented, first seizure detecting algorithm to find epileptic seizures in the offline data and to mark the epileptic seizures detected by the first seizure detecting algorithm as said true epileptic seizures, and wherein the first seizure detecting algorithm is different from the monitoring algorithm.

Clause 15: Method according to one of the clauses 1-12, wherein the true epileptic seizures are seizures detected as an epileptic seizure by a first seizure detecting algorithm which has processed the offline data and has marked the epileptic seizures detected by the first seizure detecting algorithm as said true epileptic seizures, and wherein the first seizure detecting algorithm is different from the monitoring algorithm.

Clause 16: Method according to clause 13, wherein the step x) comprises the sub-steps of
processing the offline data with a first seizure detecting algorithm to find seizures in the offline data,
processing the offline data with a second seizure to find epileptic seizures in the offline data, wherein the second seizure detecting algorithm is different from the first seizure detecting algorithm,
comparing the seizures detected by the first seizure detecting algorithm with the seizures found by the second seizure detecting algorithm and marking an event present in the offline data as a true epileptic seizure when the first seizure detecting algorithm as well as the second seizure detecting algorithm have detected this event as an epileptic seizure.

Clause 17: Method according to one of the clauses 1-12, wherein the true epileptic seizures are seizures:
detected as an epileptic seizure by a first seizure detecting algorithm which has processed the offline data and a second seizure detecting algorithm which has processed the offline data, and
marked as a said true epileptic seizure when detected by both the first seizure detecting algorithm and the second seizure detecting algorithm; and
   wherein the first seizure detecting algorithm is different from the second seizure detecting algorithm.

Clause 18: Method according to one of the clauses 16-17, wherein the first seizure detecting algorithm is a slow algorithm detecting a seizure on the basis of a change in the physiological parameter during a first period of time, wherein the second seizure detecting algorithm is a fast algorithm detecting a seizure on the basis of a change in the physiological parameter during a second period of time, and wherein the second period of time is shorter than the first period of time.

Clause 19: Method according to clause 18, wherein the first period of time is at least two times, such as at least 5 or 10 times, as large as the second period of time.

Clause 20: Method according to one of the clauses 16-19, wherein the second seizure detecting algorithm is the same as the monitoring algorithm.

Clause 21: Method according to one of the preceding clauses, comprising a step e) of setting the personalized trigger level as the trigger level of the monitoring algorithm for online use on a patient.

Clause 22: Method according to one of the preceding clauses, wherein the computer comprises a data output port configured for outputting the personalized trigger level and/or a data input port for receiving the offline data.

Clause 23: A trigger level determining apparatus for determining a personalized trigger level based on which a monitoring algorithm of an epileptic seizure detection apparatus of a patient triggers an alarm, the trigger level determining apparatus being configured for carrying out the method of any one of the clauses 1-22.

Clause 24: An epileptic seizure detection apparatus, wherein the apparatus comprises:
a physiological sensor configured for measuring a physiological parameter of a patient and generating a measurement signal representative of the measured parameter,
a processor configured to receive the measurement signal and provided with a monitoring algorithm configured to evaluate the measurement signal for presence of a trigger level and to trigger an alarm signal when the trigger level is present in the measurement signal, and
a trigger level entry configured for receiving, as the trigger level to be used in the monitoring algorithm, a personalized trigger level obtained by the method according to one of the clauses 1-22.

Clause 25: The epileptic seizure apparatus according to clause 24, wherein the apparatus further comprises a storage configured to receive and store the measurement signal as offline patient data, and wherein the processor is further configured to perform the method according to one of the clauses 1-22 using the offline data stored in the storage.

Clause 26: A computer program comprising instructions which, when the program is executed by a computer, cause the computer to carry out the steps of the method according to any one of the clauses 1-22.

Clause 27: A computer program product comprising instructions which, when the program is executed by a computer, cause the computer to carry out the steps of the method according to any one of the clauses 1-22.

Clause 28: A computer readable data carrier having stored thereon a computer program according to clause 26 or a computer program product according to clause 27.

Clause 29: A data stream which is representative of a computer program according to clause 26 or of a computer program product according to clause 27.

LIST OF REFERENCE NUMBERS

| | |
|---|---|
| 9 | screen |
| 10 | epileptic seizure detection device |
| 11 | housing |
| 12 | band |
| 13 | first physiological sensor |
| 14 | second physiological sensor |
| 15a | fastener |
| 15b | fastener |
| 16 | port |
| 17 | processor |
| 18 | storage |
| 19 | battery |
| 20 | patient |
| 21 | patient's arm |
| 100 | algorithm for method according to a first embodiment of the |
| | invention |
| 101 | set of offline data |
| 102 | start value trigger level |
| 103a | data processing algorithm |
| 103b | data processing algorithm |
| 104 | generating alarm signal when trigger level is reached |
| 105 | increase main counter by 1 when alarm signa is triggered |
| 106 | increase sub-counter by 1 when alarm signal is not associated |
| | with true epileptic seizure |
| 107 | determine extent of false alarms by comparing sub-counter and |
| | main counter |
| 108 | extent of false alarms |
| 109a | determining whether extent of false alarms is within predefined |
| | range |
| 109b | determining whether extent of false alarms is within predefined |
| | range |
| 110a | if extent < predetermined value then decrease trigger level, or if |
| | extent > predetermined value then increase trigger level |
| 110b | if extent < predetermined value then decrease trigger level, or if |
| | extent > predetermined value then increase trigger level |
| 111 | adjusted trigger level |
| 112 | outputting personalized trigger level |
| 130 | direction to continue in case YES |
| 140 | direction to continue in case NO |
| 150 | personalized trigger level |
| 200 | algorithm for method according to second embodiment of the |
| | invention |
| 201 | start value of trigger level |
| 202 | determine the extent of false alarms by testing on data base |
| 203 | determine whether extent of false alarms < predetermined value |
| 210 | increase trigger level with a measure |
| 211 | determine the extent of false alarms by testing on data base |
| 212 | determine whether extent of false alarms ≥ predetermined value |
| 220 | decrease trigger level with a measure |
| 221 | determine the extent of false alarms by testing on data base |
| 222 | determine whether extent of false alarms < predetermined value |
| 223 | Increase trigger level with a measure |
| 230 | direction to continue in case YES |
| 240 | direction to continue in case NO |
| 250 | personalized trigger level |

What is claimed is:

1. A computer implemented method for determining a personalized trigger level based on which a monitoring algorithm of an epileptic seizure detection apparatus of a patient triggers an alarm, the monitoring algorithm being configured to evaluate a measurement signal for presence of a trigger level and to trigger an alarm signal when the trigger level is detected in the measurement signal;
wherein the method uses a set of offline data representative of the patient, which set of offline data:
represents a measurement in time of at least one physiological parameter based on which the occurrence of an epileptic seizure can be determined, and
comprises a multiple of marked true epileptic seizures;
wherein the method comprises successively the steps of:
a) inputting the set of offline data as the measurement signal into the monitoring algorithm, processing the offline data with the monitoring algorithm, and, when the monitoring algorithm triggers a said alarm signal:
increasing a main-counter by 1, and
increasing a sub-counter, which counts either true alarms or false alarms, by
1 in case of a true alarm, respectively, a false alarm,
wherein a said true alarm is an alarm associated to a said true epileptic seizure and a said false alarm is an alarm not associated to a said true epileptic seizure;
b) determining an extent of false alarms by comparing the sub-counter and main counter;
c) successively and repeatedly:
decreasing the trigger level with a measure in case the extent of false alarms is below a predetermined value
or
increasing the trigger level with the measure in case the extent of false alarms is above the predetermined value,
step a), and
step b),
until the difference between the extent of false alarms and the predetermined value is within a predefined range having a lower limit and an upper limit; and
d) outputting the trigger level resulting from step c) as the personalized trigger level.

2. The method according to claim 1, wherein the lower limit is about or equal to 0.95 times the predetermined value, such as about or equal to 0.97 times the predetermined value or about or equal to 0.989 times the predetermined value.

3. The method according to claim 1, wherein the upper limit is about or equal to 1.05 times the predetermined value, such as about or equal to 1.03 times the predetermined value or about or equal to 1.011 times the predetermined value.

4. The method according to claim 1, wherein the upper limit is about or equal to the predetermined value.

5. The method according to claim 4,
wherein step c) consists of either a step c1) in case the extent of false alarms is below the predetermined value, or a step c2) in case the extent of false alarms is equal to or above the predetermined value;
wherein step c1) comprises successively and repeatedly:
decreasing the trigger level with the measure,
step a), and
step b),
until the extent of false alarms exceeds above the predetermined value; and
wherein step c2) comprises successively and repeatedly:
increasing the trigger level with the measure,
step a), and
step b),
until the extent of false alarms drops below the predetermined value.

6. The method according to claim 5, wherein in step c1), after the extent of false alarms has exceeded the predetermined value, the trigger level is lowered such that the extent of false alarms falls within the predefined range.

7. The method according to claim 1, wherein the measure is a percentage of the trigger level.

8. The method according to claim 7, wherein the measure is in the range of 0.5% to 5%, such as about 1%, of the trigger level.

9. The method according to claim 1, wherein the extent of false alarms is the percentage of alarms which is a said false alarm.

10. The method according to claim 9, wherein the predetermined value is smaller than or equal to 50%.

11. The method according to claim 10, wherein the predetermined value is in the range of 30% to 50%.

12. The method according to claim 1, wherein the extent of false alarms is the number of false alarms within a period of time.

13. The method according to claim 1, wherein the method comprises an additional step x) of processing the offline data and marking epileptic seizures found in this offline data as true epileptic seizures, and wherein the additional step x) takes place before step a).

14. The method according to claim 13, wherein the step x) uses a computer implemented, first seizure detecting algorithm to find epileptic seizures in the offline data and to mark the epileptic seizures detected by the first seizure detecting algorithm as said true epileptic seizures, and wherein the first seizure detecting algorithm is different from the monitoring algorithm.

15. The method according to claim 1, wherein the true epileptic seizures are seizures detected as an epileptic seizure by a first seizure detecting algorithm which has processed the offline data and has marked the epileptic seizures detected by the first seizure detecting algorithm as said true epileptic seizures, and wherein the first seizure detecting algorithm is different from the monitoring algorithm.

16. The method according to claim 13, wherein the step x) comprises the sub-steps of
processing the offline data with a first seizure detecting algorithm to find seizures in the offline data,
processing the offline data with a second seizure to find epileptic seizures in the offline data, wherein the second seizure detecting algorithm is different from the first seizure detecting algorithm,
comparing the seizures detected by the first seizure detecting algorithm with the seizures found by the second seizure detecting algorithm and marking an event present in the offline data as a true epileptic seizure when the first seizure detecting algorithm as well as the second seizure detecting algorithm have detected this event as an epileptic seizure.

17. The method according to claim 1, wherein the true epileptic seizures are seizures:
detected as an epileptic seizure by a first seizure detecting algorithm which has processed the offline data and a second seizure detecting algorithm which has processed the offline data, and
marked as a said true epileptic seizure when detected by both the first seizure detecting algorithm and the second seizure detecting algorithm; and
wherein the first seizure detecting algorithm is different from the second seizure detecting algorithm.

18. The method according to claim 16, wherein the first seizure detecting algorithm is a slow algorithm detecting a seizure on the basis of a change in the physiological parameter during a first period of time, wherein the second seizure detecting algorithm is a fast algorithm detecting a seizure on the basis of a change in the physiological parameter during a second period of time, and wherein the second period of time is shorter than the first period of time.

19. The method according to claim 18, wherein the first period of time is at least two times, such as at least 5 or 10 times, as large as the second period of time.

20. The method according to claim 16, wherein the second seizure detecting algorithm is the same as the monitoring algorithm.

21. The method according to claim 1, comprising a step e) of setting the personalized trigger level as the trigger level of the monitoring algorithm for online use on a patient.

22. The method according to claim 1, wherein the computer comprises a data output port configured for outputting the personalized trigger level and/or a data input port for receiving the offline data.

23. A trigger level determining apparatus for determining a personalized trigger level based on which a monitoring algorithm of an epileptic seizure detection apparatus of a patient triggers an alarm, the trigger level determining apparatus being configured for carrying out the method of claim 1.

24. An epileptic seizure detection apparatus, wherein the apparatus comprises:
a physiological sensor configured for measuring a physiological parameter of a patient and generating a measurement signal representative of the measured parameter,
a processor configured to receive the measurement signal and provided with a monitoring algorithm configured to evaluate the measurement signal for presence of a trigger level and to trigger an alarm signal when the trigger level is present in the measurement signal, and
a trigger level entry configured for receiving, as the trigger level to be used in the monitoring algorithm, a personalized trigger level obtained by the method according to claim 1.

25. The epileptic seizure apparatus according to claim 24, wherein the apparatus further comprises a storage configured to receive and store the measurement signal as offline patient data, and
wherein the processor is further configured to perform the method using the offline data stored in the storage.

26. A computer program comprising instructions which, when the program is executed by a computer, cause the computer to carry out the steps of the method according to claim 1.

27. A computer program product comprising instructions which, when the program is executed by a computer, cause the computer to carry out the steps of the method according to claim 1.

28. A computer readable data carrier having stored thereon a computer program according to claim 26 or a computer program product.

29. A data stream which is representative of a computer program according to claim 26 or of a computer program product.
